(19) United States
(12) Patent Application Publication
(10) Pub. No.: US 2008/0221658 A1
(43) Pub. Date: Sep. 11, 2008

(54) Vascular prosthesis and methods of use

(75) Inventors: Gerald Ray Martin, Redwood City, CA (US); Eric W. Leopold, Redwood City, CA (US); Michael Hogendijk, Mountain View, CA (US)

Correspondence Address: Mitchell P. Brook;LUCE, FORWARD, HAMILTON & SCRIPPS LLP, 11988 EL CAMINO REAL, SUITE 200, SAN DIEGO, CA, 92130, US

(73) Assignee: NovoStent Corporation, Mountain View, CA (US)

(21) Appl. No.: 11/716,512

(22) Filed: Mar. 9, 2007

(51) Int. Cl. A61F 2/82

(52) U.S. Cl. 623/1.12; 623/1.22; 623/1.42

(57) ABSTRACT

An implantable vascular prosthesis is provided for use in a wide range of applications wherein at least first and second helical sections having alternating directions of rotation are coupled to one another. The prosthesis is configured to conform to a vessel wall without substantially remodeling the vessel, and permits accurate deployment in a vessel without shifting or foreshortening.

### FIELD OF THE INVENTION

The present invention relates to an implantable vascular prosthesis configured for use in a wide range of applications, and more specifically, to a prosthesis having an alternating helical section.

### BACKGROUND OF THE INVENTION

Today there are a wide range of intravascular prostheses on the market for use in the treatment of aneurysms, stenoses, and other vascular irregularities. Balloon expandable and self-expanding stents are well known for restoring patency in a stenosed vessel, e.g., after an angioplasty procedure, and the use of coils and stents are known techniques for treating aneurysms.

Previously-known self-expanding stents generally are retained in a contracted delivery configuration using an outer sheath, then self-expand when the sheath is retracted. Such stents commonly have several drawbacks, for example, the stents may experience large length changes during expansion (referred to as “foreshortening” or “jumping”) and may shift within the vessel prior to engaging the vessel wall, resulting in improper placement. Another disadvantage is that after the stent is deployed it can experience longitudinal movement within the vessel (also referred to as “migration”), which can be attributed to repetitive longitudinal loading and unloading of the stent.

Additionally, repetitive loading and unloading of a stent have also been known to cause fatigue induced strut failure, which may contribute to restenosis and subsequent vessel narrowing and/or occlusion. Additionally, many self-expanding stents have relatively large delivery profiles because the configuration of their struts limits further compression of the stent. Accordingly, such stents may not be suitable for use in smaller vessels, such as cerebral vessels and coronary arteries.

For example, PCT Publication WO 00/62711 to Rivelli describes a stent comprising a helical mesh coil having a plurality of turns and including a lattice having a multiplicity of pores. The lattice is tapered along its length. In operation, the plurality of turns are wound into a reduced diameter helical shape, and then constrained within a delivery sheath. The delivery sheath is retracted to expose the distal portion of the stent and anchor the distal end of the stent. As the delivery sheath is further retracted, subsequent individual turns of the stent unwind to conform to the diameter of the vessel wall.

The stent described in the foregoing publication has several drawbacks. For example, due to friction between the turns and the sheath, the individual turns of the stent may “bunch up,” or overlap with one another, when the delivery sheath is retracted. In addition, once the sheath is fully retracted, the turns may shift within the vessel prior to engaging the vessel wall, resulting in improper placement of the stent. Moreover, because the distal portion of the stent may provide insufficient engagement with the vessel wall during subsequent retraction of the remainder of the sheath, ambiguity concerning accuracy of the stent placement may arise.

In another example, U.S. Pat. No. 5,603,722 to Phan et al. describes a stent formed of expandable strip-like segments. The strip-like segments are joined along side regions in a ladder-like fashion along offsetting side regions. A shortcoming of such a stent is that the junctions between adjacent segments are not provided with a means of addressing longitudinal loading. As a result, such a stent is susceptible to strut fracture.

In another example, U.S. Pat. No. 5,607,445 to Summers describes a balloon expandable stent. In one embodiment, the stent is constructed from a single wire that is configured so that each half of the wire is zig-zagged and curved to generally form a half-cylinder. The zig-zags of each half-cylinder are intermeshed so that they combine to form a cylindrical stent. The stent described in the foregoing publication has several drawbacks. The stent does not allow for longitudinal loading. As a result, applying a longitudinal load will cause the bends to move radially inward which will bias them into the vessel flow. Additionally, the stent design may be susceptible to fracture with repetitive loading and unloading.

In yet another example, U.S. Pat. No. 5,707,387 to Wijay describes a stent constructed from a plurality of bands, where each band is composed of a solid wire-like material formed into a closed, substantially rectangular shape. Each band is circumferentially offset from the adjacent band and adjacent bands are connected by one or more cross-tie members. This stent also has several drawbacks. The rectangular cell design does not allow for longitudinal loading because the cells are not flexible. Therefore, under a longitudinal load the apex will move out of plane and will be biased into the vessel (i.e., into the vessel flow). Secondly the stent may be susceptible to fracture with repetitive loading and unloading because of the rigid cells.

Furthermore, it generally understood that stents must be configured to provide a desired radial strength while providing adequate flexibility. However, providing additional radial strength has generally resulted in a reduction in the flexibility of the stent. Various techniques may be utilized to characterize those attributes of stents such as measuring a displacement response for a given force or determining elastic modulus. For example, radial strength may be characterized by determining the amount of force required to radially compress a stent a given distance and determining the stiffness (referred to herein as “Krad”). Alternatively, radial strength may be characterized by determining an amount of force applied linearly by opposing plates to compress a stent a given distance and determining the stiffness (referred to herein as “Kfp”).

The flexibility of a stent may be characterized by measuring the amount of force required to cause a given length of axial extension and determining the stiffness (referred to herein as “Kax”). Alternatively, the flexibility may be characterized by measuring the lateral deflection of a stent in response to a lateral force applied to a free end of the stent to determine stiffness (referred to herein as “Klat”).

A ratio of the stiffness characterizations of radial strength to flexibility may be used to provide a comparison of stents having different structures. For example, it has been found that a selected sample of currently commercially available stents generally possess Krad/Kax ratios in the range of 5-10, Krad/Klat ratios in the range of 148-184, Kfp/Kax ratios in the range of 1-6 and Kfp/Klat ratios in the range of 40-113 based on test samples each having approximately a 0.236 inch diameter, 0.906 inch length and a 0.008 inch wall thickness. It would be desirable to provide a structure that provides greater ratios so that a more optimum combination of radial strength and flexibility is provided.

When utilizing stents in interventional procedures, it may be advantageous to deliver therapeutic agents to a vessel wall via the surface of the stent. Drug eluting stents have many advantages, such as controlled delivery of therapeutic agents over an extended period of time without the need for intervention, and reduced rates of restenosis after angioplasty procedures. Typically, the drug is disposed in the matrix of a bioabsorbable polymer coated on an exterior surface of the struts of the stents, and then gradually released into a vessel wall. The quantity of the therapeutic agent provided by the stent generally is limited by the surface area of the struts. Increasing the surface area of the struts may enhance drug delivery capability, but may compromise the overall delivery profile of the stent. There therefore exists a need for a prosthesis having a reduced delivery profile and enhanced drug delivery capabilities. This is especially beneficial if other attributes such as radial strength and flexibility are not compromised.

In view of the drawbacks of previously known devices, it would be desirable to provide apparatus and methods for an implantable vascular prosthesis comprising a plurality of helical portions joined together, wherein the prosthesis is configured to be used in a wide range of applications including maintaining patency in a vessel and delivering drugs to a vessel.

It also would be desirable to provide apparatus and methods for a vascular prosthesis that is flexible enough to conform to a natural shape of a vessel without substantially remodeling the vessel.

It further would be desirable to provide apparatus and methods for a vascular prosthesis having one or more radially expanding anchors that allow for additional control over the deployment of the vascular prosthesis at a desired location within a vessel.

It still further would be desirable to provide apparatus and methods for a vascular prosthesis that has a surface area that may be selected to facilitate in-vivo delivery of therapeutic agents without adversely impacting the mechanical properties (e.g., radial strength, flexibility, etc.) of the prosthesis.

It additionally would be desirable to provide apparatus and methods for a vascular prosthesis that has a strut configuration that allows for repetitive longitudinal loading and unloading of the prosthesis.

It further would be desirable to provide apparatus and methods for a vascular prosthesis that has a structure having the ability to absorb or distribute loads.

It yet further would be desirable to provide apparatus and methods for a vascular prosthesis that has a small delivery configuration to allow the prosthesis to be used in smaller vessels.

### SUMMARY OF THE INVENTION

In view of the foregoing, it is an object of the present invention to provide apparatus and methods for an implantable vascular prosthesis comprising a plurality of helical stent portions having alternating directions of rotation joined together, wherein the prosthesis is configured to be used in a wide range of applications including, but not limited to, maintaining patency in a vessel and delivering drugs to a vessel.

It is a further object of the present invention to provide apparatus and methods for a vascular prosthesis that is flexible enough to conform to a natural shape of a vessel without substantially remodeling the vessel.

It is another object of the present invention to provide apparatus and methods for a vascular prosthesis having at least one alternating helical section that allows for controlled deployment of the vascular prosthesis at a desired location within a vessel.

It is another object of the present invention to provide apparatus and methods for a vascular prosthesis having a strut configuration that dampens the stresses associated with repetitive longitudinal loading and unloading, torsional loads, buckling and bending.

It is another object of the present invention to provide apparatus and methods for a vascular prosthesis having independent cells that absorb and/or distribute loads applied to the prosthesis.

It is a further object of the present invention to provide apparatus and methods for a vascular prosthesis that has a surface area that facilitates in-vivo delivery of therapeutic agents.

It is a further object of the present invention to provide apparatus and methods for a vascular prosthesis that has a small delivery configuration to allow the prosthesis to be used in smaller vessels.

It is a still further object of the present invention to provide apparatus and methods for a vascular prosthesis structure that provides improved ratios of radial strength to flexibility.

These and other objects of the present invention are accomplished by providing a vascular prosthesis comprising a plurality of helical portions having alternating directions of rotation that are joined together. The prosthesis is configured to engage a vessel wall and adapt to a natural curvature of the vessel. The vascular prosthesis may be used in a wide range of applications.

In a preferred embodiment, the vascular prosthesis comprises a shape memory material, such as Nitinol, and includes an alternating helical section. As used in this specification, an “alternating helical section” is formed of two or more helical portions that are joined together and have at least one change in direction of rotation of the helices. Furthermore the vascular prosthesis preferably has a Krad/Kax ratio of at least 50 and preferably has a wall thickness less than or equal to 0.010 inch and at least 15% metal area.

Prostheses of the present invention are delivered to a target vessel in a contracted state, constrained within an outer sheath, in which radially inwardly directed compressive forces are applied by the outer sheath to the anchor section(s). In the contracted state, the helical section is wound down to a reduced diameter configuration, so that adjacent turns preferably partially overlap. As an alternative, the helical section may be configured so that there is no overlap if desired. As a still further alternative, the helical section may be compressed radially to a reduced diameter configuration in addition to or in lieu of winding.

In a preferred method of operation of a prosthesis the alternating helical section is provided in its contracted state within an outer sheath and the prosthesis is fluoroscopically advanced into a selected vessel using techniques that are known in the art. The alternating helical section then is positioned adjacent a target region of a vessel, such as a stenosed region. The outer sheath then is retracted proximally to cause the first helical portion(s) of the alternating helical section to self-deploy and engage the vessel wall at the target region. Advantageously, by overlapping portions of the alternating helical section, the alternating helical section will unroll in a controlled manner. This technique ensures that the prosthesis will not shift within the vessel during deployment.

The vascular prosthesis of the present invention is flexible enough to conform to the shape of a vessel without substantially remodeling the vessel.

Additionally, the mesh configuration of the alternating helical section preferably conforms to the vasculature of the target region since each of the plurality of turns is free to assume a curved configuration substantially independently of one another. Also, because the alternating helical section of the vascular prosthesis has a ribbon-like helical structure, it may be rolled down to a contracted state with a more accurate reduced delivery profile, compared to slotted-tube stents. This feature makes the stent of the present invention especially useful for treating defects in smaller vessels, such as cerebral arteries.

In accordance with another aspect of the present invention, the plurality of turns of the alternating helical section may comprise a substantially increased surface area relative to conventional stents that have a plurality of interconnected struts. The increased surface area of the turns is particularly advantageous for localized drug delivery. The turns may be coated with a drug-laden polymer coating or, alternatively, one or more dimples or through-holes may be disposed in a lateral surface of the turns to elute drugs over an extended period of time.

Methods of using the vascular prosthesis of the present invention, for example, in the treatment of the peripheral vasculature, also are provided.

### BRIEF DESCRIPTION OF THE DRAWINGS

Further features of the invention, its nature and various advantages will be more apparent from the accompanying drawings and the following detailed description of the preferred embodiments, in which:

FIG. 1 is a schematic representation of a vascular prosthesis of the present invention in a deployed state;

FIG. 2 is a schematic representation of the vascular prosthesis of the present invention in a contracted state;

FIG. 3 is a side view of a vascular prosthesis of the present invention;

FIG. 4 is a schematic representation of the vascular prosthesis of FIG. 3 shown in a flattened configuration;

FIG. 5 is a schematic representation of another embodiment of the vascular prosthesis shown in a flattened configuration;

FIG. 6 is a schematic representation of another embodiment of the vascular prosthesis shown in a flattened configuration;

FIG. 7 is a schematic representation of another embodiment of the vascular prosthesis shown in a flattened configuration;

FIGS. 8A and 8B are side views of an apex portion of a vascular prosthesis according to the present invention;

FIGS. 9A-9C are side views of a cell of a vascular prosthesis of the present invention having alternate orientations;

FIGS. 10A-10E are side views of alternative embodiments of an apex portion of a vascular prosthesis according to the present invention;

FIGS. 11A-11E are side views of various embodiments of an apex portion of a vascular prosthesis according to the present invention;

FIG. 12 is a cross-sectional view of a delivery system suitable for use in delivering the vascular prosthesis of FIG. 3; and

FIGS. 13A-13D are side sectional views illustrating use of the vascular prosthesis in the treatment of an aneurysm;

FIG. 14 is a side view of a vascular-prosthesis of the present invention that includes distal and proximal anchors;

FIG. 15 is a schematic representation of the vascular prosthesis of FIG. 14 shown in a flattened configuration;

FIG. 16 illustrates an embodiment of a connection between an alternative embodiment of an anchor and an alternating helical section of a vascular prosthesis of the present invention; and

FIG. 17 illustrates an embodiment of a connection between an anchor and an alternating helical section of a vascular prosthesis of the present invention.

### DETAILED DESCRIPTION OF THE INVENTION

The vascular prosthesis, according to the present invention, has an alternating helix configuration that provides a more accurate reduced delivery profile than previously known devices. Additionally, the prosthesis is configured to conform to a vessel wall without substantially remodeling the vessel, to provide improved compression resistance, deployment accuracy, migration resistance and load dampening characteristics.

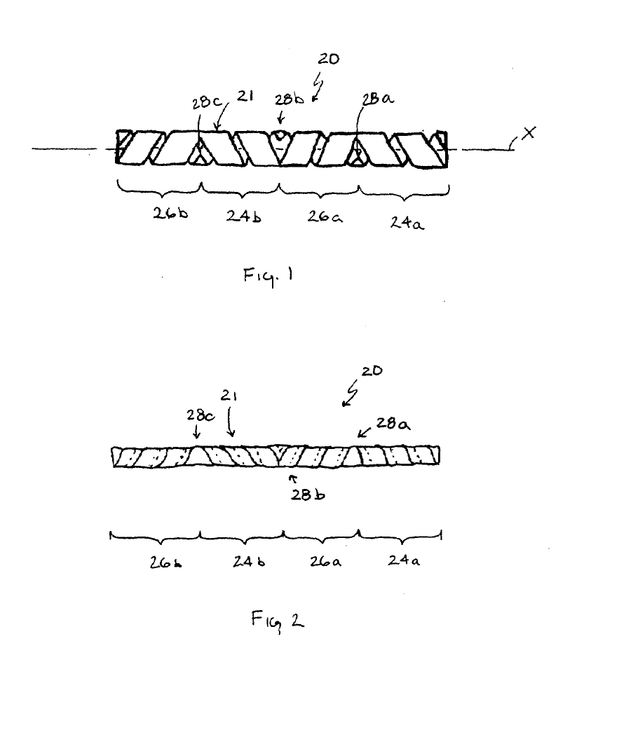

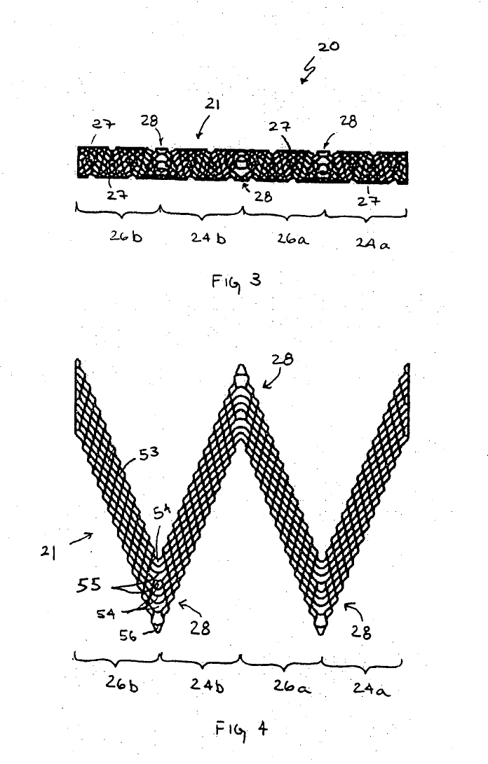

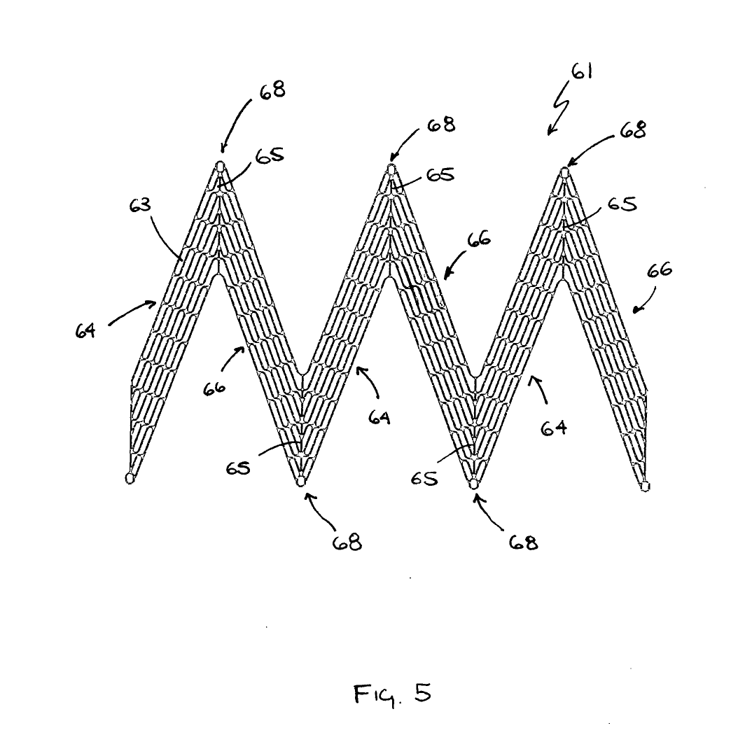

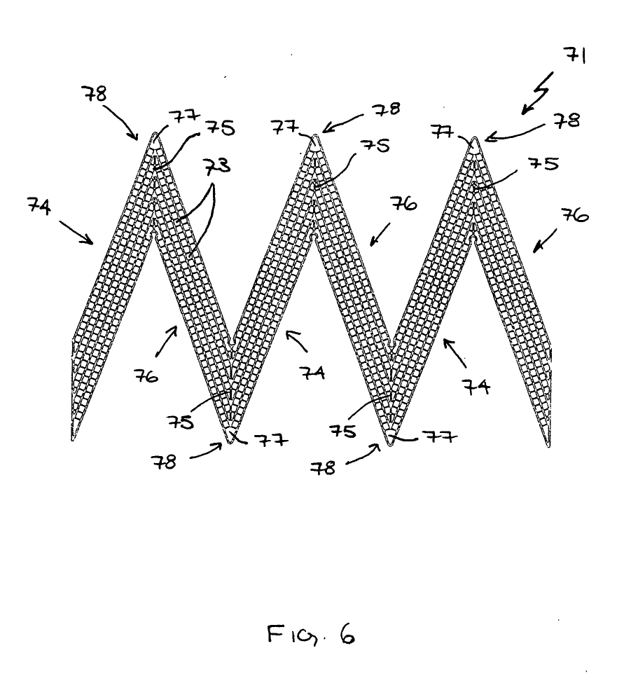

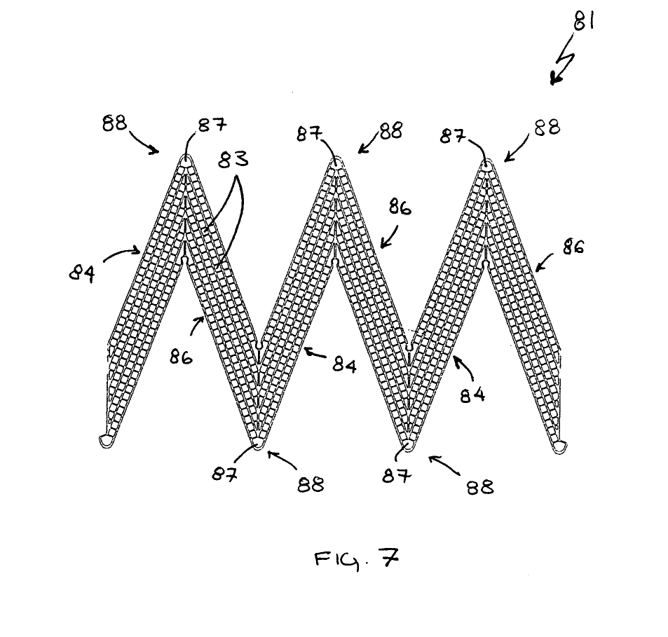

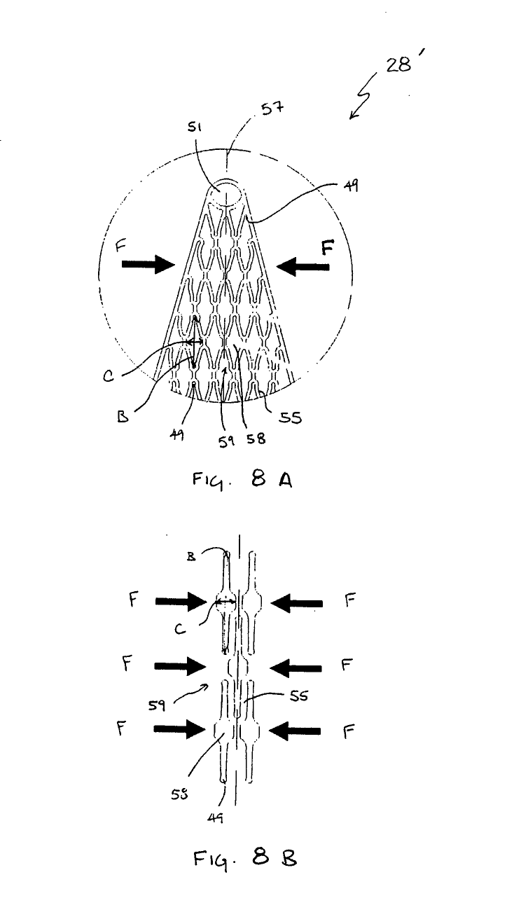

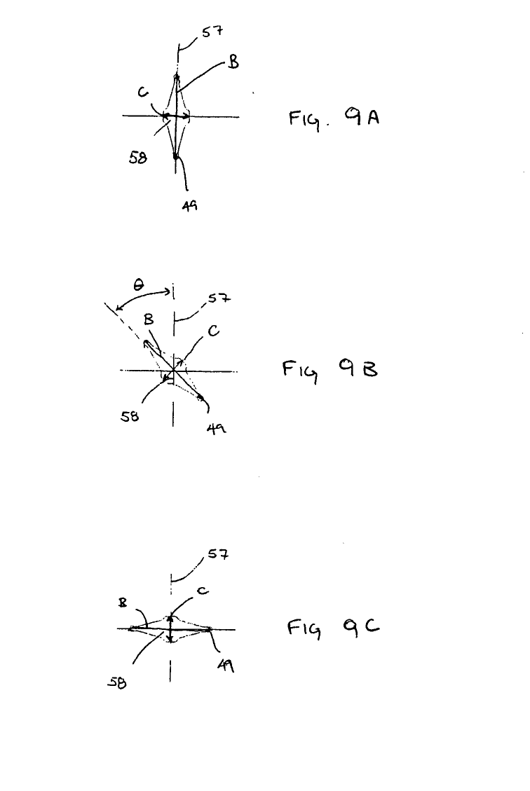

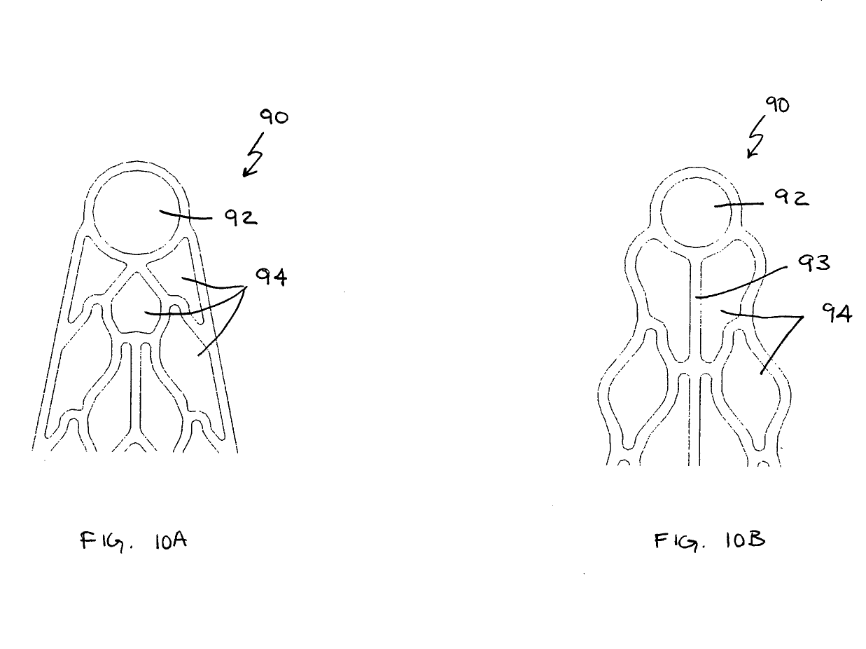

Referring now to FIGS. 1 and 2, a schematic representation of a vascular prosthesis constructed in accordance with principles of the present invention is described. Vascular prosthesis (“stent”) 20 illustratively comprises alternating helical section 21 capable of assuming contracted and deployed states. In FIG. 1, alternating helical section 21 is depicted in the deployed state.

Alternating helical section 21 is constructed from two or more helical portions having at least one change in the direction of rotation of the helices, and being joined at apex portions where the directions of rotation of adjacent helices change. In particular, first (i.e., proximal-most) helical portion 24a has a generally clockwise rotation about longitudinal axis X of prosthesis 20. Helical portion 26a adjoins the distal end of helical portion 24a at apex 28a and has a generally counter-clockwise rotation about longitudinal axis X. Helical portion 24b adjoins the distal end of helical portion 26a at apex 28b, and in turn is coupled to the proximal end of helical portion 26b at apex 28c. As a result of the alternating direction of rotation of the adjoining helical portions 24a, 26a, 24b and 26b of vascular prosthesis 20 includes three apices 28a, 28b and 28c that are oriented such that they point in alternating directions about the circumference of vascular prosthesis 20, generally in a plane that is normal to longitudinal axis X of vascular prosthesis 20. Preferably, each helical portion includes at least one full helical turn between adjacent apices. However, each helical portion may include more or less turns between adjacent apices, for example a helical portion may include 0.5-2.0 helical turns between adjacent apices.

The terminal ends of the alternating helical section may have any desired configuration. For example, as shown in FIG. 1, the terminal ends, or tails, of alternating helical section 21 are cut along a plane that is perpendicular to the longitudinal axis of vascular prosthesis 20. Alternatively, the terminal ends may be cut along any plane, such as for example parallel to the longitudinal axis. The terminal ends may end in a pointed or rounded tip or they may be truncated. As a further alternative, the width of the ribbon or mesh that forms the terminal helical portions may be varied. For example, the width of the ribbon of the terminal helical portion may taper so that it has the largest width adjacent the nearest apex and the smallest width near the terminal end. These features may be selected to provide a desired transitional flexibility at the ends of the alternating helical portion. That transitional flexibility may be used to assure that the curvature of a vessel remains smooth near the end of the stent.

A significant advantage of alternating helical section 21 as compared to other vascular prosthesis structures, is that apices 28 of alternating helical section 21 provide additional anchoring force at discrete locations along the length of alternating helical section 21. That anchoring force may be used to increase the radial force applied by the vascular prosthesis to a vessel wall as well as providing additional migration resistance. That anchoring force may be increased if desired by flaring out the ends and/or apices of the alternating helical section. Those portions may be flared outward by applying expansion and heat treatment so that those portions have a larger expanded diameter than the remainder of alternating helical section 21.

Additionally, the alternating helical configuration also allows the wall thickness of the device to be reduced because the design provides increased radial strength. More particularly, an embodiment of the alternating helical configuration has provided higher ratios of radial strength to flexibility. For example, the embodiment has provided a Krad/Kax ratio of approximately 950; a Krad/Klat ratio of approximately 1460; a Kfp/Kax ratio of approximately 640; and a Kfp/Klat ratio of approximately 990 based on a test sample having a 0.236 inch diameter, a 0.906 inch length and a 0.003 inch wall thickness. It should be appreciated that a stent may be provided that provides a Krad/Kax ratio of at least 50, or more preferably of at least 100, or even more preferably of at least 250. Similarly, a stent may be provided that provides a Krad/Klat ratio of at least 250, or more preferably of at least 500 or even more preferably of 1000. A stent may also be provided that provides a Kfp/Kax ratio of at least 50, or more preferably of at least 100 or even more preferably of at least 250. Finally, a stent may be provided that provides a Kfp/Klat ratio of at least 200, or more preferably 400 or even more preferably of at least 800.

Alternating helical section 21 preferably is formed from a solid tubular member comprised of a shape memory material, such as nickel-titanium alloy (commonly known in the art as Nitinol). However, it should be appreciated that alternating helical section 21 may be constructed from any suitable material recognized in the art. The solid tubular member then is laser cut, using techniques that are known in the art, to define a specific pattern or geometry in the deployed configuration. Preferably, alternating helical section 21 is cut from the tube so that helical portions 24a, 26a, 24b, 26b are integrally formed as a single monolithic body. However, it should be appreciated that separate helical portions may be mechanically coupled, such as by welding, soldering or installing mechanical fasteners to construct alternating helical section 21. An appropriate expansion and heat treatment then may be applied to alternating helical section 21 of vascular prosthesis 20 so that the device may be configured to self-deploy from a contracted, delivery configuration to the deployed configuration.

Referring now to FIG. 2, vascular prosthesis 20 is shown in the contracted, delivery configuration, wherein alternating helical section 21 is in the contracted, reduced diameter state. Alternating helical section 21, however, is placed in the contracted state by winding helical portions 24, 26 about longitudinal axis X. In FIG. 2, apices 28a and 28c may be temporarily engaged to the inner shaft of a delivery catheter, and the shaft is rotated while apex 28b and the distal and proximal ends of alternating helical section 21 are held stationary.

Consequently, apices 28a and 28c are tightly wound onto the shaft of the delivery catheter and the remainder of each helical portion 24, 26 is wound against the shaft so that each turn of each portion 24, 26 overlaps an adjacent turn. For example, in some embodiments, approximately ⅔ of a layer is overlapped by the next layer. As a result, apex 28b and the distal and proximal ends of alternating helical section 21 are located furthest radially outward on the rolled alternating helical section 21. The overlap of the turns of helical portions 24, 26 are indicated by dashed lines in FIG. 2. The overlapping turns of alternating helical section 21 thus secure apices 28a and 28c when vascular prosthesis 20 is disposed within a delivery system.

Referring now to FIGS. 3 and 4, an embodiment of vascular prosthesis 20, constructed in accordance with principles of the present invention, is described. It should be appreciated that FIG. 4 is a schematic view of vascular prosthesis 20 as it would appear if it were flattened. The components of vascular prosthesis 20 are identical to those depicted in FIGS. 1 and 2 and identical reference numbers are employed in the following description.

Alternating helical section 21 preferably comprises a helical mesh configuration including two or more helical portions 27. Helical portions 27 may include multiplicity of openings 53, 54, 56 of different shapes and sizes. The shape, size and orientation of any particular opening is selected to provide a desired response to longitudinal loads and also may be dependent upon the location of the openings within the mesh structure. The shape, size and orientation of the openings may also be selected to provide desired deployment, unwrapping, radial force and surface area coverage characteristics.

As shown in FIG. 4, alternating helical section 21 includes diamond-shaped openings 53 of generally equal size through the majority of each helical portion 24, 26.

A wide variety of openings may be employed at apices 28a, 28b and 28c, where the helical portions adjoin adjacent helical portions. The openings may have any shape and/or size desired. Some designs include diamond, polygon, circles, elipses, elongated diamonds, etc. In addition, the openings of apices 28 need not be symmetric with respect to a centerline of apex 28. It should be appreciated that the size, shape and orientation of any of the openings may be selected so that in the deployed state some struts may bow radially outward or inward so that they interlock with adjacent, overlapping openings.

In FIG. 4, each apex includes plurality of openings 54 and one tip opening 56 that forms a tip of the respective apex, which may be triangular as shown. Openings 54 are defined by struts 55 that extend between adjacent helical portions 24, 26.

Referring to FIG. 5, another embodiment of alternating helical section 61 will be described. As with the previously described embodiments, alternating helical section includes a plurality of helical portions 64, 66 having alternating directions of rotation that are joined at apices 68. Each of helical portions 64, 66 is constructed from a helical mesh that defines openings 63. Openings 63 are generally Z-shaped openings that are relatively larger than openings 53 of the previously described embodiments. As a result of that opening design, each unit cell design is larger which allows the flexibility to be tailored so that the vascular prosthesis has desired longitudinal and radial flexibility. Such flexibility may be used to improve radial force applied by and/or fatigue strength of the prosthesis or to aid deployment. As shown, openings 63 are generally elongate along the length of the helical portion and narrow along the longitudinal axis of alternating helical section 61. Additionally, at each apex 68 a strut 65 extends generally along a center line of apex which, in an actual configuration of the vascular prosthesis extends generally circumferentially about the prosthesis at apex 68. In addition, the edges of helical portions 64, 66 are straight which may be used to ease deployment. It is noted that a curved, or wavy, edge such as that shown in FIGS. 3 and 4 may provide alternate benefits such as improved metal coverage and cell interlocking. For example, relative sliding of the portions of a wavy alternating helical section may provide a ratchet effect so that the overlapping portions may be incrementally and temporarily interlocked during deployment.

In another embodiment, shown in FIG. 6, alternating helical section 71 includes helical portions 74, 76. Each of helical portions 74, 76 is constructed from mesh that defines generally square openings 73. Similar to the previously described embodiment, alternating helical section 71 includes straight edges and a strut 75 that extends generally along a center line of apex 78. Additionally, alternating helical section 71 includes tip openings 77 that are generally triangular in shape. The shape and size of tip openings 77 may be tailored to provide a desired interface with a delivery device and/or to provide desired flexibility at apex 78.

In a still further embodiment, shown in FIG. 7, alternating helical section 81 includes helical portions 84, 86 constructed from mesh having square openings 83 similar to the previous embodiment. However, the configuration of apices 88 is different. In particular struts located in apices 88 were removed to provide added flexibility in that region. In addition, tip openings 87 are rounded rather than triangular as compared to those shown in FIG. 6. Those changes may be used to reduce fatigue at apices 88 and/or to simplify deployment or loading onto a delivery system.

Referring to FIGS. 8A and 8B, an alternative strut configuration for the stent and particularly the apices of the vascular prosthesis is described. Apex 28′ is constructed with struts 55 that form plurality of cells 59 defining elongate openings 58. Elongate openings 58 allow cells 59 to be compressed in response to longitudinal loads (shown by arrows F) placed on vascular prosthesis 20.

In addition, tip aperture 51, or eyelet, is included in apex 28′. Apertures 51 are provided so that apex 28′ may be easily coupled to a delivery device, as will be described in greater detail below. As shown, aperture 51 is generally elliptical, but it should be appreciated that the shape of aperture 51 will generally correspond to the structure of the intended delivery device.

Elongate openings 58 each generally have major axis B corresponding to the longest distance across opening 58 and minor axis C corresponding to the shortest distance across opening 58. Referring to FIG. 8B, a portion of apex 28′ of FIG. 8A is shown with cells 59 compressed under the influence of longitudinal force F. Elongate openings 58 are oriented so that major axis B of each opening 58 is parallel with center line 57 of apex 28′ and minor axis C of each opening 58 is perpendicular to center line 57. During compression minor axis C is reduced while major axis B remains generally unchanged. As a result, the longitudinal load may be dampened by compression of the mesh structure of vascular prosthesis 20.

Elongate openings 58 preferably are shaped to reduce stress concentration. In the present embodiment, elongate openings 58 are generally diamond-shaped with rounded corners 49 at the junctions of adjacent struts 55. It should be appreciated that elongate openings may be any elongate shape. The size, shape and orientation of cells 59 on either side of center line 57 are shown generally identical. With such a configuration, dampening occurs equally from both sides of center line 57 when a longitudinal load is applied. However, it should be appreciated that the dampening characteristics of vascular prosthesis 20 may be tailored by including cells having different size, shape and/or orientation on either or both sides of center line 57. It is also noted that the helical portions of the stent provide a significant level of additional dampening of forces, including torsional and buckling. Furthermore, it should be appreciated that the apices included throughout vascular prosthesis 20 need not be identical and may be configured to provide differing dampening characteristics throughout vascular prosthesis 20.

In addition, the orientation of openings 58 with respect to center line 57 and the longitudinal axis of vascular prosthesis may be selected to further control load dampening characteristics. Referring to FIGS. 9A-9C various exemplary orientations of openings 58 will be described. Openings 58 may be oriented so that major axis B is parallel to center line 57 as shown in FIG. 9A. In that orientation, cells 59 are configured so that longitudinal loading of a vascular prosthesis causes longitudinal compression. Openings 58 also may be oriented so that major axis B is angled with respect to center line 57. For example, as shown in FIG. 9B, opening 58 is oriented so that major axis B is rotated by angle θ from center line 57, which is approximately 45 degrees. In another embodiment, opening 58 may be oriented so that major axis B is perpendicular to center line 57 and as a result longitudinal forces may be redirected circumferentially through apex 28. It should be appreciated that the orientations of openings 58 also may be utilized throughout helical portions 24, 26 for openings 53 to distribute stress as desired. Additionally, a portion of openings 53, 54, 56, 58 may be replaced by fully covered portions, if desired, to provide additional surface area to interface a vessel wall, such as for drug delivery.

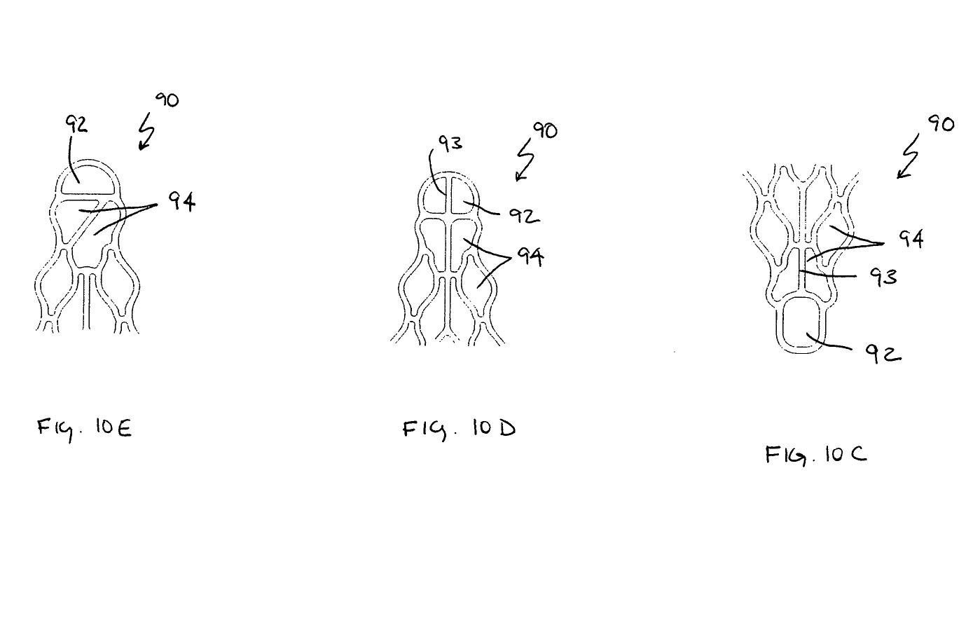

Referring now to FIGS. 10A-10E, further alternative configurations of openings of apices 90 are described. Apices 90 may include openings having a variety of shapes and sizes. In one embodiment, shown in FIG. 10A, apex 90 includes a relatively large circular tip opening 92 and a plurality of irregularly shaped openings 94 adjacent tip opening 92 that are generally symmetric with respect to a centerline of apex 90. In one example, tip opening 92 is circular and has a diameter of approximately 0.056 inches.

In another embodiment, shown in FIG. 10B, the size of tip opening 92 is reduced when compared to the previously described embodiment and a circumferential strut 93 extends through apex 90 to tip opening 92. Circumferential strut 93 may be employed to alter the flexibility of apex 90 in addition to or instead of altering the thickness of the struts of apex 90. In a similar embodiment, shown in FIG. 10C, tip opening 92 is generally a square.

In another embodiment, shown in FIG. 10D, circumferential strut 93 extends through tip opening 92 of apex 90, thereby defining a pair of tip openings 92 which are generally semi-circular. Circumferential strut 93 may be used to further tailor the flexibility of apex 90 and to increase the radial strength at the tip of apex 90.

In yet another embodiment, shown in FIG. 10E, tip of apex 90 includes a semi-circular tip opening 92 and adjacent asymmetric openings 94. In particular, apex 90 includes two openings immediately adjacent tip opening 92 that have different sizes and shapes so that a strut extends between the openings at an angle with respect to both a centerline of apex 90 and the longitudinal axis of the vascular prosthesis. Such asymmetric cells may be provided to provide desired flexibility and/or to aid in deployment. It will be appreciated that any of the various embodiments of the apices described herein may be used at any location along a vascular prosthesis. It should be appreciated that the tips of the apices may be enlarged so that they form a larger opening than shown in the previous embodiments. Such an enlarged tip may be provided to ease coupling with a delivery device or to reduce the stress placed on a vessel wall by the tip.

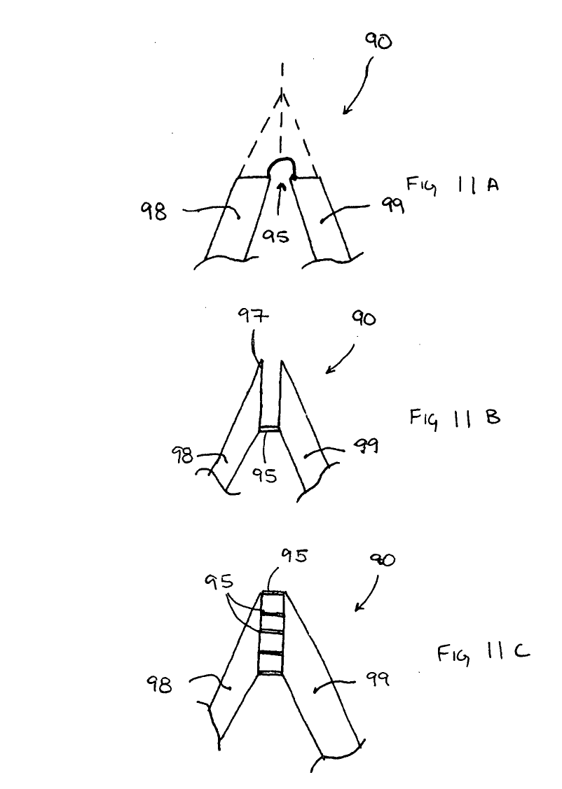

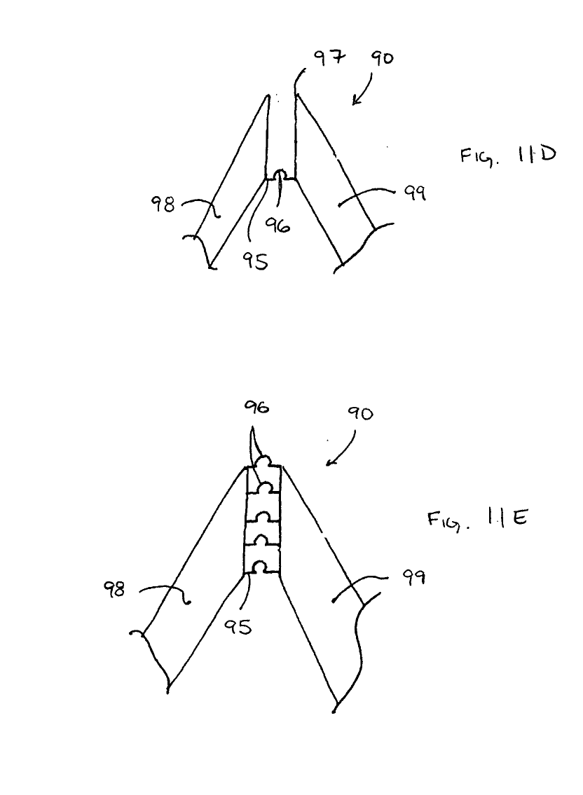

Referring now to FIGS. 11A-11E, alternative configurations of apices 90 are described. As shown in FIG. 11A, single strut 95 may couple adjacent helical portions 98, 99 and helical portions 98, 99 may be configured so that an edge of each helical portion 98, 99 aligns with strut 95. This configuration provides additional torsional flexibility between adjacent helical portions 98, 99.

In the further alternative embodiment of FIG. 11B, helical portions 98, 99 may be coupled by single strut 95 and each of helical portions 98, 99 may extend further circumferentially past strut 95. In such an embodiment, helical portions 98, 99 may terminate in spaced apart tips 97. In addition to providing additional torsional flexibility and longitudinal load dampening, tips 97 may be designed to engage retaining features of a delivery system.

In a still further alternative of FIG. 11C, plurality of struts 95 extend between helical portions 98, 99 thereby defining plurality of openings 94. Helical portions 98, 99 terminate in tips 97 that are coupled by strut 95 in a spaced relationship. This configuration provides torsional flexibility between adjacent helical portions 98, 99, while limiting longitudinal compression of openings 94. It should be appreciated that struts 95 may have any desired orientation. For example, struts may be parallel, angled or perpendicular with respect to the longitudinal axis of the vascular prosthesis.

Struts 95 may include hinge members 96, as depicted in FIGS. 11D and 11E. Hinge members 96 may be provided to alter the flexibility of struts 95, as desired. The embodiment illustrated in FIG. 11D generally corresponds to the embodiment previously described with reference to FIG. 11B, however hinge member 96 has been added to single strut 95 extending between helical portions 98, 99. Similarly, the embodiment illustrated in FIG. 11E generally corresponds to the embodiment previously described with reference to FIG. 11C, however hinge members 96 are included on each of the plurality of struts 95. The configurations shown in FIGS. 11C and 11E also improve deployment stability, and in particular linear placement stability.

Hinge members 96 may be any shape to alter the flexibility of strut 95. In FIG. 11E, hinge members 96 comprise U-shaped portions of struts 95. It should be appreciated that hinge members 96 may be any shape desired, such as S or Z-shaped portions. Additionally, hinge members 96 may be constructed from a material different than the remainder of strut 95.

As will be apparent to one skilled in the art, the configuration of the alternating helical sections depicted herein is merely for illustrative purposes. Any combination of covered portions and openings of any shape and size may be provided along the helical portions, as desired. Alternatively, one or more helical portions may be completely solid, such that the openings are omitted entirely from that portion. As a further alternative the entire alternating helical section may be covered so that the device may be used as a stent graft. In such an embodiment, materials such as ePTFE and Dacron are examples of materials that may be used to cover the alternating helical section.

As will be apparent to those skilled in the art, a combination of solid regions and openings may be provided along the length of the alternating helical section, for example, to selectively increase surface area and drug delivery capabilities along the alternating helical section, or to influence flow dynamics within a vessel.

It will be appreciated that different drug delivery modalities may be used in conjunction with the vascular prosthesis of the present invention. For example, vascular prosthesis may include one or more dimples and/or through holes that may have a therapeutic agent disposed therein. As a further alternative, a therapeutic agent may be incorporated into the any of the openings previously described above. As a still further alternative, a therapeutic agent may be disposed in the matrix of a bioabsorbable polymer coated on any portion of the vascular prosthesis, and the drug may be gradually released into a localized region of a vessel wall.

One or more of the helical portions also may be selectively coated with an elastomeric polymer, such as polyurethane. The elastomeric polymer may partially or fully cover the selected portions. As a further alternative, the covering material may be included only in the openings of the mesh structure so that it fills the openings without increasing the overall diameter of the struts. For example, the elastomeric polymer may be disposed on a portion of the circumference of the alternating helical section, e.g., to reduce blood flow into a sac of the aneurysm. Additionally, a therapeutic agent may be disposed on the elastomeric polymer to increase the working surface area of the alternating helical section. Alternatively, the therapeutic agent may be disposed directly on the alternating helical section, either with or without the use of an elastomeric polymer.

The therapeutic agent may include, for example, antiplatelet drugs, anticoagulant drugs, antiproliferative drugs, agents used for purposes of providing gene therapy to a target region, or any other agent, and may be tailored for a particular application. Radiopaque markers (not shown) also may be selectively disposed on any portion of vascular prosthesis including in the vicinity of the therapeutic agents to facilitate alignment of the therapeutic agents with a target site of a vessel wall. Advantageously, higher doses of such agents may be provided using the vascular prosthesis of the present invention, relative to previously known coils or stents having interconnected struts, due to the increased surface area associated with the alternating helical section.

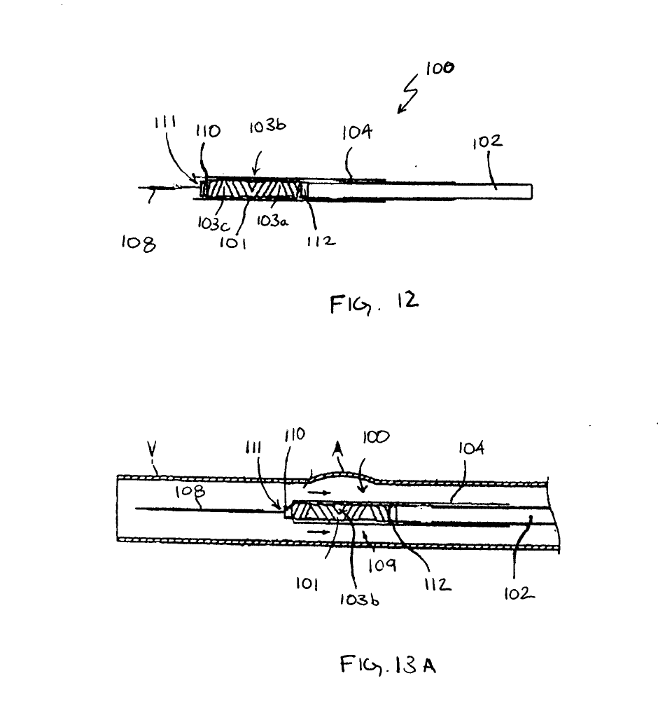

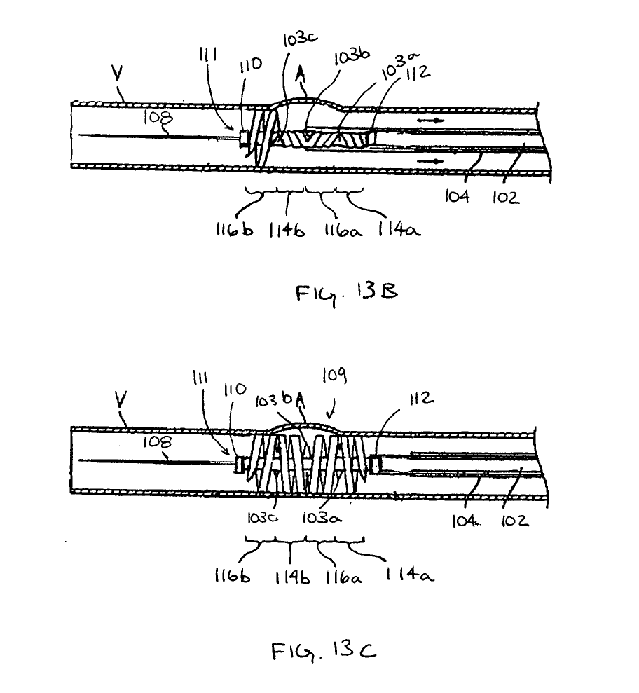

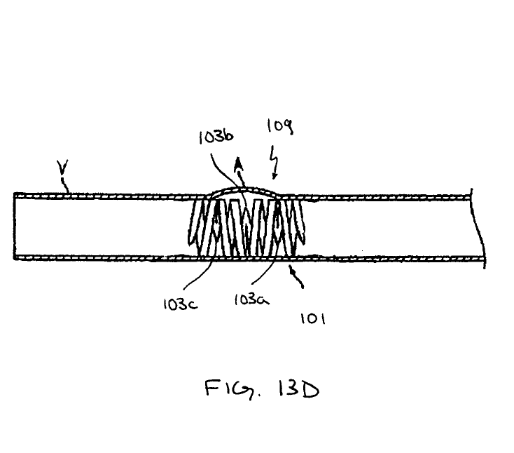

In operation, the overlap of portions of the alternating helical section when it is in the contracted state and the number of helical portions, causes alternating helical section 101 to deploy in a unique sequence, as will be described in greater detail below with reference to FIGS. 13A-13D. Advantageously, the order of deployment of the portions of alternating helical section 101 alleviates drawbacks associated with the prior art such as the tendency of the turns of the helical section to jump or shift during deployment and also results in the location of deployment being more easily controlled. Another benefit is that deployment of discrete segments may be more easily controlled. Additionally, the alternating helical section may be balloon expandable. In particular, the structure allows a user to post dilate discrete sections with a balloon. For example, a user may expand a selected portion of the device adjacent a specific apex.

In FIG. 12, a delivery system 100 suitable for use in delivering a vascular prosthesis of the present invention is described. Delivery system 100 comprises catheter body 102, outer sheath 104, and a lumen dimensioned for the passage of guidewire 108. Catheter body 102 preferably includes distal marker 111 and stop 110 located adjacent the distal end of alternating helical section 101 and proximal stop 112 located adjacent the proximal end of alternating helical section 101.

Distal stop 110 may comprise a raised ledge on catheter body 102 so that the distal end of alternating helical section 101 bears on the ledge to prevent relative movement between alternating helical section 101 and catheter body 102 in the distal direction. Alternatively, distal stop 110 may comprise a plurality of raised pins or knobs that prevent relative motion between alternating helical section 101 and catheter body 102 parallel to the longitudinal axis. Proximal stop 112 also may comprise a raised ledge, pins or knobs on catheter body 102, and both distal and proximal stops 110 and 112 may be radiopaque, so as to be visible under a fluoroscope and provide a radiopaque marker. It should be appreciated that any portion of the delivery device or vascular prosthesis may include one or more radiopaque markers.

Vascular prosthesis 109 is collapsed onto catheter body 102 by winding alternating helical section 101 around catheter body 102. In order to wind alternating helical section 101 on catheter body 102, apices 103a and 103c may be temporarily coupled to catheter body 102 and the remainder of alternating helical section 101 is wound around catheter body 102 until it is collapsed as shown in FIG. 12.

After alternating helical section 101 is wound on catheter body 102, outer sheath 104 is advanced distally over catheter body 102 to capture alternating helical section 101 between catheter body 102 and outer sheath 104.

Referring to FIG. 13A, in operation, guidewire 108 is percutaneously and transluminally advanced through a patient's vasculature, using techniques that are known in the art. Guidewire 108 is advanced until a distal end of guidewire 68 is positioned distal of aneurysm A, which is situated in vessel V. Delivery system 100, having vascular prosthesis 109 contracted therein, then is advanced over guidewire 108 through the central lumen of catheter body 102. Delivery system 100 preferably is advanced under fluoroscopic guidance until distal marker 111 is situated distally to aneurysm A and alternating helical section 101 and apex 103b are situated adjacent to the aneurysm.

Once alternating helical section 101 is located adjacent to aneurysm A, outer sheath 104 is retracted proximally to cause alternating helical section to deploy until outer sheath 104 is retracted to proximal stop 112.

Referring to FIGS. 13B and 13C, after the distal end of alternating helical section 101 is secured distal of aneurysm A, outer sheath 104 is further retracted proximally to allow alternating helical section 101 to continue to expand and deploy to its predetermined deployed shape. During proximal retraction of outer sheath 104, the stent rotates within the artery, or may be manually rotated through rotation of the delivery system, to enable alternating helical section 101 to unwind. Because central portions of the alternating helical section are over-wrapped, rotation of catheter body 102 is not required for the alternating helical section to expand.

As outer sheath 104 is further retracted, the turns of alternating helical section 101 unwind and engage and conform to an inner wall of vessel V in a controlled manner. Helical portion 116b expands as outer sheath 104 is moved proximal of the distal end of alternating helical section 101. Helical portion 116b is not able to expand until the distal end of outer sheath 104 is moved proximal of apex 103b because alternating helical section 101 is wound so that apex 103b is located radially outward (i.e., outer-wrapped) and overlaps the adjacent helical portions. After the distal end of outer sheath 104 is moved sufficiently proximal of apex 103b, helical portions 114b and 116a are allowed to expand. For example, inner-wrapped apices, such as apices 103a and 103c, are constrained by the adjacent helical portions 114 and 116 and as a result those apices remain constrained until sufficient exposure of the stent occurs to release the helical portions, thereby creating a controlled release of the stent. Finally, after sheath 104 is moved proximal of the proximal end of alternating helical section 101, helical portion 114a is able to expand, as illustrated in FIG. 13C.

Proximal movement of outer sheath 104 may be halted once the distal edge of outer sheath 104 is substantially aligned with proximal stop 112 to allow alternating helical section 101 to expand. It will be appreciated that because of the sequence of deployment of alternating helical section 101, the location of the deployed alternating helical section 101 may be easily controlled and the problems encountered in previous systems (e.g., stent jumping) may be avoided.

When vascular prosthesis 109 is fully deployed, delivery system 100 is proximally retracted over guidewire 108 and withdrawn from the patient's vessel, and guidewire 108 is removed. After removal of delivery system 100 and guidewire 108, vascular prosthesis 109 remains deployed, as shown in FIG. 13D.

In the present invention, the partial overlap of portions of alternating helical section 101 reduce the surface area that is available to frictionally engage an inner surface of outer sheath 104. Furthermore, the sequence of deployment of the alternating helices included in alternating helical section 101 also assures that the prosthesis remains properly located during deployment. Advantageously, the helical portions of the alternating helical section will be accurately deployed within vessel V, with substantially no proximal or distal shifting or foreshortening of the prosthesis with respect to the vessel as the outer sheath of the delivery device is retracted.

It should be appreciated that the furthest proximal and the furthest distal helical portions may be configured so that the proximal and distal tips of the alternating helical section are either inner-wrapped or outer-wrapped as desired. As shown in FIGS. 13A-D both tips may be outer-wrapped. As further alternative, one tip may be outer-wrapped and the other inner-wrapped or both tips may be inner-wrapped. It will be appreciated that inner-wrapped portions of the alternating helical section generally require expansion of complimentary portions of the alternating helical section before the entire prosthesis is capable of expansion.

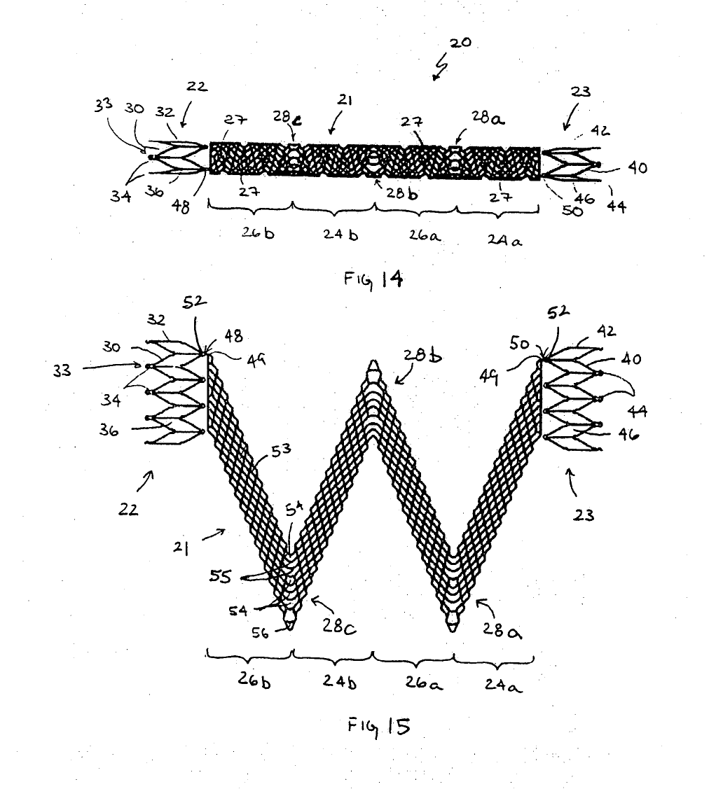

Referring to FIGS. 14 and 15, another embodiment of vascular prosthesis 20 is shown, which includes optional distal and proximal anchor sections 22, 23. Distal anchor section 22 preferably is a tubular mesh structure that is coupled to a distal end of alternating helical section 21. In particular, distal anchor section 22 includes a pair of concentrically aligned zig-zag rings 30 that are spaced from one another and coupled by struts 32. Struts 32 extend between corresponding apices 34 of rings 30 and are oriented parallel to a longitudinal axis of vascular prosthesis 20. Apices 34 may comprise one or more radiopaque markers 33 such as a radiopaque marker band or coating. As a result, rings 30 and struts 32 combine to define a plurality of openings 36 shaped as parallelograms, thereby forming a tubular mesh. The tubular mesh preferably is formed by laser cutting a solid tube.

Distal anchor section 22 preferably is formed from a solid tubular member comprising a shape memory material, such as nickel-titanium alloy, which is laser cut, using techniques that are known in the art, to a desired deployed configuration. Preferably, distal anchor section 22 is cut from the tube so that rings 30 and struts 32 are formed as a single monolithic body. However, it should be appreciated that distal anchor section 22 may be constructed from separate rings 30 and struts that are mechanically coupled in a secondary operation, such as by welding, soldering or employing a mechanical fastener, such as a rivet. An appropriate heat treatment then may be applied so that distal anchor section 22 may be configured to self-deploy radially outward from a contracted, delivery configuration to a deployed configuration in conjunction with alternating helical section 21, described above. Alternatively, distal anchor section 22 may be configured to be balloon expandable.

Proximal anchor section 23 also preferably has a tubular mesh construction. Proximal anchor section 23 includes pair of concentrically aligned zig-zag rings 40 that are spaced from one another and coupled by struts 42. Struts 42 extend between corresponding apices 44. Apices 44 may comprise one or more radiopaque markers 43 such as a radiopaque marker, band or coating. Rings 40 are oriented parallel to longitudinal axis X of vascular prosthesis 20. Rings 40 and struts 42 combine to define a plurality of openings 46 shaped as parallelograms. Similar to distal anchor section 22, the tubular mesh structure of proximal anchor section 23 preferably is formed by laser cutting a solid tube. Proximal anchor section 23 may be constructed in the same manner described above with respect to distal anchor section 22. Alternatively, proximal anchor section 23 also may be constructed to be balloon expandable.

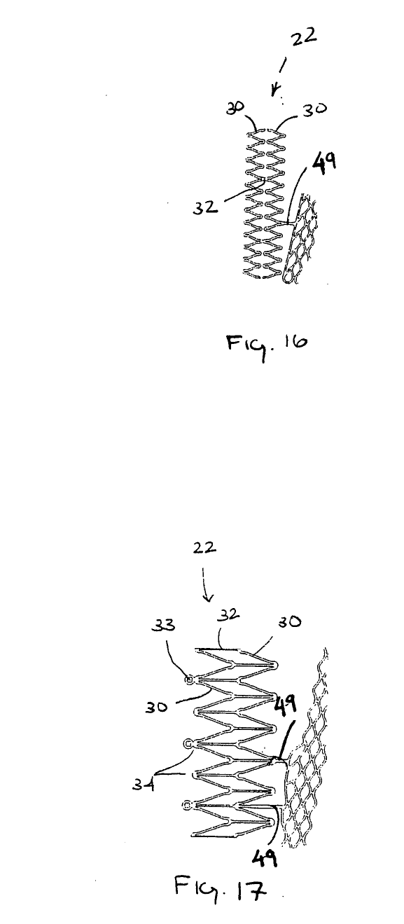

Moreover, distal anchor section 22 and proximal anchor section 23 may have different constructions. Although distal anchor section 22 and proximal anchor section 23 as described above are identical, they alternatively may have different zig-zag or cell structures or deployment modes (e.g., self-expanding at the distal end and balloon expandable at the proximal end). For example, anchor sections 22, 23 may be constructed as a single zig-zag ring. As a further alternative, anchor sections 22, 23 may be configured so that openings 36, 46 have shapes other than parallelograms, e.g., openings 36, 46 may be shaped as diamonds or any other polygonal shape, circles or ellipses. Furthermore, although anchor sections 22, 23 are illustrated as including struts 32, 42 extending between each set of corresponding apices, struts 32, 42 may extend between fewer sets of corresponding apices. For example, as shown in FIG. 16, struts may extend between relatively few apices. In addition, the distance between the zig-zag rings of anchor sections 22, 23 may also be selected to provide an anchor section of any desired length.

Furthermore, the outer edges of anchor sections 22, 23 may be biased so that the proximal-most edge of anchor section 23 and the distal-most edge of anchor section 22 expand further radially outward than with respect to longitudinal axis X than the remainder of the anchor section. This configuration may be useful to increase radial outward force upon a patient's vessel and thus improve anchoring of vascular prosthesis 20 within the vessel. Such a biased configuration may be established by heat-treating a shape memory material using techniques that are known in the art.

Distal anchor section 22 is coupled to the distal end of alternating helical section 21 at junction 48. Similarly, proximal anchor section 23 is coupled to the proximal end of alternating helical section 21 at junction 50. Preferably, junctions 48, 50 are formed from a strut of alternating helical section 21 that extends from that section and is coupled to a portion of the adjacent zig-zag rings 30, 40 of the respective anchor section 22, 23.

Junctions 48, 50 may comprise one or more radiopaque markers 52 such as a radiopaque marker band or coating. Radiopaque marker 52 facilitates positioning of junctions 48, 50 at a desired longitudinal position within a patient's vessel, and further facilitates alignment of vascular prosthesis 20 at a desired axial orientation within the vessel. For example, radiopaque markers 52 may be used to orient alternating helical section 21 so that a desired lateral surface of alternating helical section 21 deploys to overlay the diseased vessel segment.

It will be apparent to those skilled in the art that junctions 48, 50 may comprise other strut arrangements to connect distal anchor section 22 and proximal anchor section 23 to alternating helical section 21. For example, more than one strut may extend from alternating helical section 21 to a respective anchor 22, 23.

Various alternate junction configurations will be described which may be used to couple distal anchor section 22 and/or proximal anchor section 23 to alternating helical section 21. As described above and as shown in FIGS. 14-17, anchors 22, 23 are preferably coupled to alternating helical section 21 by one or more struts 49 that may extend generally parallel to longitudinal axis X of the vascular prosthesis. However, it is noted that a perpendicular strut is possible and would provide improved flexibility when loaded on the delivery system. Struts 49 may be any desired length and may extend to any portion of the adjacent anchor. For example, struts 49 may extend to an apex 34 of anchor 22 or any other portion of anchor 22. In addition, struts 49 may extend from any portion of alternating helical section 21 near an end of the section. For example, as shown in FIG. 15, strut 49 extends from a tip of alternating helical section 21, and as shown in FIGS. 16 and 17, struts 49 extend from a portion of alternating helical section 21 away from the tip.

Referring to FIG. 17, anchor 22 is coupled to alternating helical section 21 at a location spaced from the tip of alternating helical section 21. Anchor 22 is also coupled to alternating helical section 21 by a plurality of struts that extend generally parallel to the longitudinal axis of the vascular prosthesis. Moreover, the length of the struts is increased to increase the distance between anchor 22 and alternating helical section 21.

In one preferred embodiment, alternating helical section 21, distal anchor section 22 and proximal anchor section 23 are integrally formed as a single monolithic body, such as by laser cutting all three components from a single tube. In such a construction of vascular prosthesis 20, the struts extending from alternating helical section 21 that form junctions 48, 50 also may form struts 32, 42 of the respective anchor section 22, 23. Alternatively, anchor sections 22, 23 may be manufactured separately from alternating helical section 21 and mechanically coupled in a subsequent process, such as by soldering, welding, installing mechanical fasteners (e.g., rivets) or other means, as will be apparent to one skilled in the art. A further advantage over the above-mentioned publications is that the configuration of the alternating helical section provides dampening characteristics for longitudinal, torsional and buckling forces applied to the vascular prosthesis.

Although a method of treating diseased vessels has been described, it will be apparent from the method described herein that the vascular prosthesis may be used in a variety of procedures. For example, vascular prosthesis also may be used in general stenting procedures, for example, to maintain patency in a vessel after a carotid angioplasty procedure, or may be used as an intravascular drug delivery device, or may be used in other applications apparent to those skilled in the art.

In accordance with another aspect of the present invention, the vascular prosthesis of the present invention is configured to be flexible enough to substantially conform to the shape of vessel V without causing the vessel to remodel. In particular, the alternating direction of rotation of the helical portions of the alternating helical section allow for increased flexibility of the prosthesis.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. A vascular prosthesis for implantation in a body vessel having a vessel wall, the vascular prosthesis comprising:
an alternating helical section comprising three or more helical portions, wherein a first helical portion has a direction of rotation about a longitudinal axis of the prosthesis opposite to that of a second helical portion, and a third helical portion that has a direction of rotation about the longitudinal axis the same as the first helical portion, the first and second helical portions adjoined at a first apex and the second and third helical portions adjoined at a second apex.

2. The vascular prosthesis of claim 1, wherein the alternating helical section includes an even number of helical portions having a first direction of rotation and an even number of helical portions having a second direction of rotation, wherein the adjoining helical portions define an odd number of apices.

3. The vascular prosthesis of claim 1, wherein the alternating helical section includes an even number of helical portions having a first direction of rotation and an odd number of helical portions having a second direction of rotation, wherein the adjoining helical portions define an even number of apices.

4. The vascular prosthesis of claim 1, wherein the helical portions are joined by at least one strut.

5. The vascular prosthesis of claim 1, wherein the helical portions are joined by at least one hinge.

6. The vascular prosthesis of claim 1, wherein at least one helical portion is a helical mesh.

7. The vascular prosthesis of claim 6, wherein the helical mesh defines a plurality of diamond shaped apertures.

8. The vascular prosthesis of claim 6, wherein the helical mesh defines a plurality of Z-shaped apertures.

9. The vascular prosthesis of claim 1, further comprising a therapeutic agent disposed on or in a portion of the alternating helical section.

10. The vascular prosthesis of claim 1, further comprising a polymer disposed on or in a portion of the alternating helical section.

11. The vascular prosthesis of claim 10, wherein the polymer is configured to elute a therapeutic agent.

12. The vascular prosthesis of claim 1, wherein the alternating helical section comprises a shape memory material.

13. The vascular prosthesis of claim 1, further comprising a radially expanding anchor section joined to a first end of the alternating helical section.

14. The vascular prosthesis of claim 13, further comprising a second radially expanding anchor section joined to a second end of the alternating helical section.

15. The vascular prosthesis of claim 14, wherein the alternating helical section, the first anchor section and the second anchor section each are capable of assuming a contracted state suitable for transluminal insertion into the body vessel and a deployed state wherein the helical section, first anchor section and second anchor section are configured to engage the vessel wall.

16. The vascular prosthesis of claim 13, wherein the shape memory material is a nickel titanium alloy.

17. The vascular prosthesis of claim 16, wherein the alternating helical section and the anchor section are separately formed and then coupled together.

18. The vascular prosthesis of claim 13, wherein the alternating helical section and the anchor section are integrally formed.

19. The vascular prosthesis of claim 1, wherein the portions of the helical portions overlap when the alternating helical section is in a contracted state.

20. A vascular prosthesis for implantation in a body vessel having a vessel wall, the vascular prosthesis comprising:
an alternating helical section comprising first, second and third helical portions, wherein the first and third helical portions have a direction of rotation about a longitudinal axis of the prosthesis opposite to that of the second helical portion, the first and second helical portions coupled at a first apex and the second and third helical portions coupled at a second apex,
wherein all of the helical portions are constructed from struts that form a mesh having a plurality of apertures.

21. The vascular prosthesis of claim 20, further comprising a first radially self-expanding distal anchor section joined to a first end of the alternating helical section.

22. The vascular prosthesis of claim 21, further comprising a second radially self-expanding proximal anchor section joined to a second end of the alternating helical section.

23. The vascular prosthesis of claim 20, wherein the alternating helical section includes an even number of helical portions having a first direction of rotation and an even number of helical portions having a second direction of rotation.

24. The vascular prosthesis of claim 20, wherein the alternating helical section comprises a shape memory material.

25. The vascular prosthesis of claim 24, wherein the shape memory material is a nickel titanium alloy.

26. The vascular prosthesis of claim 21, wherein the alternating helical section and the anchor section are separately formed and then coupled together.

27. A method of deploying a vascular prosthesis, comprising:
advancing a guidewire to a diseased vessel segment;
advancing a delivery system having a vascular prosthesis loaded therein over the guidewire to the diseased vessel segment, wherein the vascular prosthesis includes an alternating helical section, the alternating helical section comprising first, second and third helical portions, wherein the first and third helical portions have a direction of rotation opposite to that of the second helical portion, and wherein the vascular prosthesis is wound onto a catheter body of the delivery system when the vascular prosthesis is in a contracted configuration;
retracting an outer sheath of the delivery system proximally to expose the alternating helical section, the alternating helical section expanding to a deployed configuration; and
retracting the delivery system from the vessel.

28. The method of deploying a vascular prosthesis of claim 27, wherein the vascular prosthesis further comprises an anchor section coupled to an end of the alternating helical section, the method further comprising retracting the outer sheath proximally to allow the anchor section to expand to a deployed configuration prior to retracting the delivery system from the vessel.

29. A vascular prosthesis for implantation in a body vessel having a vessel wall, the vascular prosthesis comprising:
a body portion that provides a Krad/Kax ratio of at least 50.

30. The vascular prosthesis of claim 29, the body has a wall thickness of less than or equal to 0.010 inch.

31. The vascular prosthesis of claim 29, wherein the body portion has a metal surface area relative to the vessel surface area covered by the vascular prosthesis of at least 15 percent.

32. The vascular prosthesis of claim 29, wherein an expanded diameter of the vascular prosthesis is at least three times the contracted delivery diameter of the vascular prosthesis.

33. The vascular prosthesis of claim 29, wherein the body portion provides a Krad/Kax ratio of at least 100.

34. The vascular prosthesis of claim 33, wherein the body portion provides a Krad/Kax ratio of at least 250.
